(19) United States
(12) Patent Application Publication
(10) Pub. No.: US 2020/0353446 A1
(43) Pub. Date: Nov. 12, 2020

(54) CATALYST COMPOSITION FOR THE OXIDATIVE COUPLING OF METHANE USING A SILVER PROMOTER

(71) Applicant: Sabic Global Technologies, B.V., Bergen op zoom (NL)

(72) Inventors: Wugeng LIANG, Sugar Land, TX (US); Luanyi Elizabeth LI, Sugar Land, TX (US); Hector PEREZ, Sugar Land, TX (US); Dick NAGAKI, Sugar Land, TX (US); Vidya Sagar Reddy SARSANI, Sugar Land, TX (US); David WEST, Sugar Land, TX (US)

(21) Appl. No.: 16/864,900

(22) Filed: May 1, 2020

Related U.S. Application Data
(60) Provisional application No. 62/845,892, filed on May 10, 2019.

(51) Int. Cl. B01J 23/50; B01J 37/08; C07C 2/84

(52) U.S. Cl. CPC B01J 23/50; B01J 37/088; C07C 2523/10; C07C 2523/50; C07C 2/84

(57) ABSTRACT

The invention relates to a catalyst composition, suitable for producing ethylene and other commercially high value C₂₊ hydrocarbons from methane. The composition contains a silver promoted mixed metal catalyst composition comprising at least two rare earth elements and an alkaline rare earth metal element. The catalyst composition has high catalyst activity and enables oxidative coupling of methane reactions to be conducted at a low reactor temperature while retaining sufficient catalyst selectivity. The invention further provides a method for preparing such a catalyst composition and a process for producing C₂₊ hydrocarbons, using such a catalyst composition.

### CROSS REFERENCE TO RELATED APPLICATIONS

This application claims priority to U.S. Provisional Application No. 62/845,892 filed May 10, 2019 and entitled “Catalyst Composition for the Oxidative Coupling of Methane Using a Silver Promoter,” the disclosure of which is hereby incorporated by reference in its entirety.

### FIELD OF INVENTION

The invention relates to the field of catalyst compositions and more particularly to catalyst compositions used for the oxidative coupling of methane (OCM).

### BACKGROUND

Ethylene is one of the most important building blocks in the chemical industry and maximizing its production, while retaining desired operating profits through technology advancements, is important for all ethylene producers. Methane is a widely available feedstock having high calorific value, and if oxidatively coupled, in presence of suitable methane coupling catalysts, commercially high value chemicals such as ethylene and other C₂₊ hydrocarbons, can be produced sustainably at high production margins. However, for reactions where methane is used as a reactant, activating methane for undergoing a chemical reaction may be a challenging proposition owing to its thermodynamic stability. Keeping such an objective in mind, catalyst development for the industrial production of ethylene and other C₂₊ hydrocarbons from methane, is an area of research, which has attracted considerable attention from both industry and academia, aimed at removing certain limitations associated with OCM catalysts and their effective use.

One such limitation is the temperature at which a catalyst may be subjected in a reactor tube, for effecting the complete conversion of methane and oxygen during an OCM reaction. At low reactor temperature or generally at low OCM temperature conditions, oxygenated byproducts are formed, which lowers catalyst productivity and desired product yield. Further, at such temperature conditions, oxygen conversion is severely impacted as methane is not sufficiently activated, leading to large amounts of unreacted methane in product mixtures, which limits the utility of such product mixtures and increases the overall operational expenditure. On the other hand, at high reactor temperature or generally at high OCM temperature conditions, catalyst degradation may occur along with the formation of undesirable deep oxidation byproducts of carbon monoxide and carbon dioxide, thereby affecting catalyst selectivity performance and reducing carbon efficiency towards formation of useful chemicals. In addition, there are certain inherent challenges posed by OCM processes, such as high heat generation during an exothermic OCM reaction, which requires limiting the methane conversion to a low value, in order to avoid a runaway reaction. Previous attempts to solve this problem by providing a cooled multi-tubular reactor, have proved to be commercially unfeasible.

An alternate way to manage the heat generated during an OCM process, is by effectively utilizing the heat generated during an OCM reaction to activate methane. To achieve this goal, a thin catalyst bed may be used, which requires catalyst systems with high catalyst activity. With such catalyst systems, an adiabatic reactor may be used, resulting in significant reduction in capital and operational expenditure (see for example, Chemical Engineering Journal, 2017, vol. 328, 484). Additional advantages of using such high activity catalyst systems, are that of low catalyst loading required for effecting the oxidative coupling reaction, resulting in improved plant operation and energy utilization. Unfortunately, designing such catalyst systems have proved to be challenging in the past, as catalyst activity and catalyst selectivity are generally of opposite attribute. Further, for OCM reactions, high input feed temperature is required for activating the relatively inert methane feed prior to coupling, leading to drawbacks associated with high reactor temperature as described above. Thus, it is evident that the OCM reaction needs to be conducted at a suitable temperature, which is sufficient for complete conversion of methane to commercially high value chemicals. Although OCM catalysts have been described in various literature publications, such as the published patent applications WO2015101345A1 (Published: July 2015) or EP3194070A2 (Published: July 2017), such catalyst systems can still be further improved upon, in terms of their selectivity and activity performance especially at a low reactor temperature. Other technical solutions as proposed in the US published patent application US20170014807, describe silver promoted oxidative coupling of methane with specific mixed metal oxide compositions. However, as will be shown by way of this disclosure and specifically by way of Example 6 of this disclosure, performance of silver promoted OCM catalysts, may still be further improved upon.

Thus, from the foregoing reasons, there remains a need to develop a catalyst composition for the oxidative coupling of methane, having one or more benefits of having high catalyst activity even when subjected to a relatively low oxidative coupling temperature conditions, while retaining sufficient catalytic selectivity for producing C₂₊ hydrocarbon products.

### SUMMARY

The invention relates to a composition, comprising a catalyst represented by a general formula (I): (Ag_{z}AEₐRE1_{b}RE2_{c}AT_{d}Oₓ) wherein, (i) ‘Ag’ represents silver; (ii)‘AE’ represents an alkaline earth metal; (iii) ‘RE’ represents a first rare earth element; (iv) ‘RE2’ represents a second rare earth element; and (v) ‘AT’ represents a third rare earth element ‘RE3’, or a redox agent selected from antimony, tin, nickel, chromium, molybdenum, tungsten; wherein, ‘a’, ‘b’, ‘c’, ‘d’ and ‘z’ represents relative molar ratio; wherein ‘a’ is 1; ‘b’ ranges from about 0.1 to about 10; ‘c’ ranges from about 0.01 to about 10; ‘d’ ranges from 0 to about 10; ‘z’ ranges from about 0.01 to about 1; ‘x’ balances the oxidation state; wherein, the first rare earth element, the second rare earth element and the third rare earth element, are different.

In some embodiments of the invention, the relative molar ratio ‘z’ ranges from about 0.04 to about 0.18. In some embodiments of the invention, the relative molar ratio ‘a’ is 1, the relative molar ratio ‘b’ is 0.9, the relative molar ratio ‘c’ is 0.7, the relative molar ratio ‘d’ is 0.1, and the relative molar ratio ‘z’ ranges from about 0.043 to about 0.093.

In some embodiments of the invention, the alkaline earth metal ‘AE’ is selected from the group consisting of magnesium, calcium, strontium, barium, and combinations thereof. In some embodiments of the invention, the first rare earth element (RE1), the second rare earth element (RE2), and the third rare element (RE3) are each independently selected from the group consisting of lanthanum, scandium, cerium, praseodymium, neodymium, promethium, samarium, europium, gadolinium, yttrium, terbium, dysprosium, holmium, erbium, thulium, ytterbium, lutetium, and combinations thereof. In some embodiments of the invention, alkaline earth metal ‘AE’ is strontium, first rare earth element ‘RE’ is lanthanum, second rare earth element ‘RE2’ is neodymium, third rare earth element ‘RE3’ is ytterbium. In some embodiments of the invention, the catalyst has a formula represented by Ag_{0.046}Sr_{1.0}La_{0.9}Nd_{0.7}Yb_{0.1}Oₓ, wherein the relative molar ratio ‘z’ is 0.046, the relative molar ratio ‘a’ is 1, the relative molar ratio ‘b’ is 0.9, the relative molar ratio ‘c’ is 0.7, and the relative molar ratio ‘d’ is 0.1. In some embodiments of the invention, the catalyst has a formula represented by Ag_{0.091}Sr_{1.0}La_{0.9}Nd_{0.7}Yb_{0.1}Oₓ, wherein the relative molar ratio ‘z’ is 0.091, the relative molar ratio ‘a’ is 1, the relative molar ratio ‘b’ is 0.9, the relative molar ratio ‘c’ is 0.7, and the relative molar ratio ‘d’ is 0.1. In some embodiments of the invention, the catalyst has a formula represented by Ag_{0.083}Sr_{1.0}La_{0.9}Nd_{0.7}Yb_{0.1}Oₓ, wherein the relative molar ratio ‘z’ is 0.083, the relative molar ratio ‘a’ is 1, the relative molar ratio ‘b’ is 0.9, the relative molar ratio ‘c’ is 0.7, and the relative molar ratio ‘d’ is 0.1. In some aspects of the invention, the composition has a 90% oxygen conversion temperature (T(90%)° C.) ranging from about 200° C. to about 700° C., when the composition is used in a process for producing C₂₊ hydrocarbon mixture product from methane and oxygen. In some aspects of the invention, the composition has a 90% oxygen conversion temperature (T(90%)° C.) ranging from about 300° C. to about 500° C., when the composition is used in a process for producing C₂₊ hydrocarbon mixture product from methane and oxygen.

In some aspects of the invention, the composition has a 90% oxygen conversion temperature (T(90%)° C.) ranging from about 2% to about 50% lower than 90% oxygen conversion temperature (T(90%)° C.) of a silver-free catalyst composition having the formula (AEₐRE1_{b}RE2_{c}AT_{d}Oₓ) where (i) ‘AE’ represents an alkaline earth metal; (ii) ‘RE1’ represents a first rare earth element; (iii) ‘RE2’ represents a second rare earth element; and (iv) ‘AT’ represents a third rare earth element ‘RE3’, or a redox agent selected from antimony, tin, nickel, chromium, molybdenum, tungsten; wherein, ‘a’, ‘b’, ‘c’, and ‘d’ represents relative molar ratio; wherein ‘a’ is 1; ‘b’ ranges from 0.1 to 10; ‘c’ ranges from about 0.01 to about 10; ‘d’ ranges from 0 to about 10; ‘x’ balances the oxidation state; wherein, the first rare earth element, the second rare earth element and the third rare earth element, are different.

In some embodiments of the invention, the composition has a C₂₊ hydrocarbon selectivity ranging from about 70% to about 88% of total product formed, when the composition is used in a process for producing C₂₊ hydrocarbon mixture product from methane and oxygen. In some embodiments of the invention, the composition has a C₂₊ hydrocarbon selectivity ranging from about 98% to about 105% of C₂₊ hydrocarbon selectivity of the silver-free catalyst composition having the formula (AEₐRE1_{b}RE2_{c}AT_{d}Oₓ) where (i) ‘AE’ represents an alkaline earth metal; (ii) ‘RE1’ represents a first rare earth element; (iii) ‘RE2’ represents a second rare earth element; and (iv) ‘AT’ represents a third rare earth element ‘RE3’, or a redox agent selected from antimony, tin, nickel, chromium, molybdenum, tungsten; wherein, ‘a’, ‘b’, ‘c’, and ‘d’ represents relative molar ratio; wherein ‘a’ is 1; ‘b’ ranges from 0.1 to 10; ‘c’ ranges from about 0.01 to about 10; ‘d’ ranges from 0 to about 10; ‘x’ balances the oxidation state; wherein, the first rare earth element, the second rare earth element and the third rare earth element, are different. In some embodiments of the invention, the composition achieves a methane conversion ranging from about 10% to about 50%, when the composition is used in a process for producing C₂₊ hydrocarbon mixture product from methane and oxygen.

In some embodiments of the invention, a method for preparing the composition comprising the catalyst of the present invention is provided, where the method comprises: (i) forming an aqueous catalyst precursor solution comprising a silver agent and a precursor mixture comprising an alkaline earth metal compound and at least two rare earth metal compounds; (ii) drying the aqueous catalyst precursor solution at a temperature of at least 90° C. and forming a dried catalyst precursor mixture; and (iii) calcining the dried catalyst precursor mixture for at least 5 hours at a temperature of at least 650° C. and forming the composition. In some embodiments of the invention, the method of preparing the composition comprising the catalyst, further comprises calcining the precursor mixture and forming a calcined precursor mixture. In embodiments of the invention, the silver agent is selected from the group consisting of silver nanoparticles, silver nanowires, silver salts and combinations thereof. In some embodiments of the invention, the silver agent is a silver nanoparticle. In some other embodiments of the invention, the silver agent is a silver salt. In some aspects of the invention, the invention relates to a process for producing a C₂₊ hydrocarbon mixture product comprising: (a) introducing a feed mixture comprising methane and oxygen in a reactor containing the catalyst of the present invention; (b) subjecting the feed mixture to a methane coupling reaction under conditions suitable to produce the C₂₊ hydrocarbon mixture product; and (c) recovering the C₂₊ hydrocarbon mixture product after removing unconverted methane and steam from the C₂₊ hydrocarbon mixture product. In some embodiments of the invention, methane to oxygen ratio ranges from about 2:1 to about 15:1. In some embodiments of the invention, the feed mixture comprising methane and oxygen is introduced in the reactor at a feed temperature of less than 400° C. In some embodiments of the invention, the C₂₊ hydrocarbon mixture product is produced at a reactor temperature ranging from about 300° C. to about 800° C.

Other objects, features and advantages of the invention will become apparent from the following figures, detailed description, and examples. It should be understood, however, that the figures, detailed description, and examples, while indicating specific embodiments of the invention, are given by way of illustration only and are not meant to be limiting. Additionally, it is contemplated that changes and modifications within the spirit and scope of the invention will become apparent to those skilled in the art from this detailed description. In further embodiments, features from specific embodiments may be combined with features from other embodiments. For example, features from some specific embodiments may be combined with features from any of the other embodiments. In further embodiments, additional features may be added to the specific embodiments described herein.

### BRIEF DESCRIPTION OF THE DRAWINGS

For a more complete understanding, reference is now made to the following descriptions taken in conjunction with the accompanying drawings, in which:

FIG. 1 is a graphical representation of the oxygen conversion of the inventive catalyst composition prepared under Example 2, as an embodiment of the invention, under different reactor temperature.

FIG. 2 is a graphical representation of the oxygen conversion of the inventive catalyst composition prepared under Example 4, as an embodiment of the invention, under different reactor temperature.

FIG. 3 is a graphical overview of the catalyst activity and C₂₊ hydrocarbon selectivity obtained from the inventive examples and the control compositions including the comparative Example 6.

### DETAILED DESCRIPTION

The invention is based, in part, on the discovery that a composition containing a catalyst, can be used for the oxidative coupling of methane with one or more benefits of having high catalyst activity and sufficient product selectivity even when subjected to a relatively low oxidative coupling temperature conditions. Advantageously, the catalyst composition of the present invention enables a feed mixture comprising methane and oxygen, to be fed in a reactor at a temperature thereafter effect methane coupling reaction at a temperature, which is previously unseen for the OCM reactions. The catalyst composition, is formulated by combining silver metal promoters having specific dimension and at specific proportion, with mixed metal oxides containing at least rare earth metals and an alkaline earth metal.

The following includes definitions of various terms and phrases used throughout this specification.

The terms “about” or “approximately” or “substantially” are defined as being close to as understood by one of ordinary skill in the art. In some non-limiting embodiments the terms are defined to be within 1%, preferably, within 0.1%, more preferably, within 0.01%, and most preferably, within 0.001%.

The terms “wt. %”, “vol. %”, or “mol. %” refers to a weight, volume, or molar percentage of a component, respectively, based on the total weight, the total volume, or the total moles of material that includes the component. In a non-limiting example, 10 moles of a particular component present in a 100 moles of a material is 10 mol. % of component.

The use of the words “a” or “an” when used in conjunction with the term “comprising,” “including,” “containing,” or “having” in the claims or the specification may mean “one,” but it is also consistent with the meaning of “one or more,” “at least one,” and “one or more than one.”

The words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The method of the invention can “comprise,” “consist essentially of,” or “consist of” particular ingredients, components, compositions, etc., disclosed throughout the specification.

Any numerical range used through this disclosure shall include all values and ranges there between unless specified otherwise. For example, a boiling point range of 50° C. to 100° C. includes all temperatures and ranges between 50° C. and 100° C. including the temperature of 50° C. and 100° C.

The term “overall C₂₊ hydrocarbon” or “C₂₊ hydrocarbon mixture product” as used in this disclosure means the hydrocarbon products produced using the inventive catalyst composition and having at least two carbon atoms and includes ethylene, ethane, ethyne, propene, propane, and C₄-C₅ hydrocarbons. The term oxidative coupling of methane or “OCM” as referred or used through this disclosure means the oxidative coupling of methane or the reaction of methane and oxygen, for the production of C₂₊ hydrocarbons from methane.

The term “catalyst activity” as used throughout this disclosure means catalyst activity for the reaction of methane with oxygen whether or not it is expressly stated as such, unless expressly stated otherwise. The catalyst activity is proportional to the percentage of oxygen conversion at a specific temperature for example, at a temperature ranging from about 300° C. to about 800° C. and can be determined using a gas chromatograph and calculated using the equation: k=−Ln(1−XO₂/100), (Eqn I), where XO₂ is the oxygen conversion rate. For the purposes of this invention, oxygen conversion can be measured by comparing the oxygen concentration at the outlet and inlet of an oxidative coupling of methane reactor, such a reactor being a 2.3 mm ID quartz tube reactor having a feed mixture flow rate adjusted from about 40 sccm and a catalyst loading of 20 mg. Alternatively, a parameter which serves as a convenient proxy for catalyst activity is the temperature at which 90% of the oxygen conversion takes place, herein represented as (T(90%)° C.). In this way, lower values of (T(90%)° C.) indicate higher catalyst activity than do higher values of (T(90%)° C.).

The term “redox agent” as used herein means substances or elements capable of undergoing or promoting or supporting both oxidation or reducing reactions.

The term “selectivity” to a desired product or products refers to how much desired product was formed divided by the total products formed, both desired and undesired. For purposes of the disclosure herein, the selectivity to a desired product is a % selectivity based on moles converted into the desired product. Further, for purposes of the disclosure herein, a Cₓ selectivity (e.g., C₂ selectivity, C₂₊ selectivity, etc.) can be calculated by dividing a number of moles of carbon (C) from CH₄ that were converted into the desired product (e.g., C_{C2H4}, C_{C2H6}, etc.) by the total number of moles of C from CH₄ that were converted (e.g., C_{C2H4}, C_{C2H6}, C_{C2H2}, C_{C3H6}, C_{C3H8}, C_{C4}s, C_{CO2}, C_{CO}, etc.). C_{C2H4}=number of moles of C from CH₄ that were converted into C₂H₄; C_{C2H6}=number of moles of C from CH₄ that were converted into C₂H₆; C_{C2H2}=number of moles of C from CH₄ that were converted into C₂H₂; C_{C3H6}=number of moles of C from CH₄ that were converted into C₃H₆; C_{C3H8}=number of moles of C from CH₄ that were converted into C₃H₈; C_{C4}s=number of moles of C from CH₄ that were converted into C₄ hydrocarbons (C₄s); C_{CO2}=number of moles of C from CH₄ that were converted into CO₂; C_{CO}=number of moles of C from CH₄ that were converted into CO; etc. A C₂₊ hydrocarbon selectivity (e.g., selectivity to C₂₊ hydrocarbons) refers to how much C₂H₄, C₃H₆, C₂H₂, C₂H₆, C₃H₈, C₅s and C₄s were formed divided by the total product formed which includes C₂H₄, C₃H₆, C₂H₂, C₂H₆, C₃H₈, C₄s, C₅s, C_{n′s} CO₂ and CO. Accordingly, a preferred way of calculating C₂₊ hydrocarbon selectivity will be by using the equation:

$$( \frac{( \begin{matrix}2 {C}_{C 2 H 4} + 2 {C}_{C 2 H 6} + 2 {C}_{C 2 H 2} + 2 {C}_{C 3 H 6} + \\ 3 {C}_{C 3 H 8} + 4 {C}_{C 4 \text{?}} + 5 {C}_{C 5 \text{?}} + {nC}_{Cn \text{?}}\end{matrix} )}{( \begin{matrix}2 {C}_{C 2 H 4} + 2 {C}_{C 2 H 6} + 2 {C}_{C 2 H 2} + 2 {C}_{C 3 H 6} + 3 {C}_{C 3 H 8} + \\ 4 {C}_{C 4 \text{?}} + 5 {C}_{C 5 \text{?}} + {nC}_{Cn \text{?}} + {C}_{CO 2} + {C}_{CO}\end{matrix} )} ) \times 100$$

$$\text{?} \text{indicates text missing or illegible when filed}$$

Specifically, a high C₂₊ hydrocarbon selectivity will signify increased formation of useful C₂₊ hydrocarbon products over that of undesirable byproducts.

The invention provides for a composition, containing a catalyst of the present invention, comprising a silver promoter and a mixed metal oxide containing an alkaline earth metal element and at least two rare earth metal elements. Particularly, some aspects of the invention relates to a composition, comprising a catalyst represented by a general formula (I): (Ag_{z}AEₐRE1_{b}RE2_{c}AT_{d}Oₓ) wherein, (i) ‘Ag’ represents silver; (ii)‘AE’ represents an alkaline earth metal; (iii) ‘RE1’ represents a first rare earth element; (iv) ‘RE2’ represents a second rare earth element; and (v) ‘AT’ represents a third rare earth element ‘RE3’, or a redox agent selected from antimony, tin, nickel, chromium, molybdenum, tungsten; wherein, ‘a’, ‘b’, ‘c’, ‘d’ and ‘z’ represents relative molar ratio; wherein ‘a’ is 1; ‘b’ ranges from about 0.1 to about 10, alternatively from about 0.5 to about 8, alternatively from about 0.9 to about 2; ‘c’ ranges from about 0.01 to about 10, alternatively from about 0.07 to about 1, alternatively from about 0.07 to about 0.8; ‘d’ ranges from 0 to about 10, alternatively from about 0.1 to about 5; ‘z’ ranges from about 0.01 to about 1, alternatively from about 0.04 to about 0.1, alternatively 0.07 to about 0.09; ‘x’ balances the oxidation state; wherein, the first rare earth element (RE1), the second rare earth element (RE2) and the third rare earth element (RE3), are different. In some aspects of the invention, ‘Ag’ is silver derived from silver nanoparticles. In some aspects of the invention, ‘Ag’ is silver derived from silver salts.

The term “different” as used herein means that each of the rare earth elements are different chemical elements. In some embodiments of the invention, the alkaline earth metal (AE) is selected from the group consisting of magnesium, calcium, strontium, barium, and combinations thereof. In some preferred embodiments of the invention, the alkaline earth metal (AE) is strontium. In some embodiments of the invention, the first rare earth element (RE1), the second rare earth element (RE2), and the third rare element (RE3) are each independently selected from the group consisting of lanthanum, scandium, cerium, praseodymium, neodymium, promethium, samarium, europium, gadolinium, yttrium, terbium, dysprosium, holmium, erbium, thulium, ytterbium, lutetium, and combinations thereof. In some preferred embodiments of the invention, the relative molar ratio ‘a’ is 1, the relative molar ratio ‘b’ is 0.9, the relative molar ratio ‘c’ is 0.7, the relative molar ratio ‘d’ is 0.1, and the relative molar ratio ‘z’ ranges from about 0.043 to about 0.093, alternatively from about 0.044 to about 0.09, alternatively from about 0.05 to about 0.08. In some preferred embodiments of the invention, the first rare earth (RE1) element is lanthanum and is present at a relative molar ratio ‘b’ of 0.9. In some preferred embodiments of the invention, the second rare earth element (RE2) is neodymium (Nd) and is present at a relative molar ratio of ‘c’ of 0.7. Without wishing to be limited by any particular theory, the incorporation of stable rare earth metal oxides imparts catalytic stability to the composition and mitigates risks of catalyst deactivation during the oxidative coupling reaction. In some embodiments of the invention, the catalyst has a formula represented by Ag_{0.046}Sr_{1.0}La_{0.9}Nd_{0.7}Yb_{0.1}Oₓ wherein the relative molar ratio ‘z’ is 0.046, the relative molar ratio ‘a’ is 1, the relative molar ratio ‘b’ is 0.9, the relative molar ratio ‘c’ is 0.7, and the relative molar ratio ‘d’ is 0.1 and wherein ‘Ag’ is silver derived from silver nanoparticles. In some embodiments of the invention, the catalyst has a formula represented by Ag_{0.091}Sr_{1.0}La_{0.9}Nd_{0.7}Yb_{0.1}Oₓ, wherein the relative molar ratio ‘z’ is 0.091, the relative molar ratio ‘a’ is 1, the relative molar ratio ‘b’ is 0.9, the relative molar ratio ‘c’ is 0.7, and the relative molar ratio ‘d’ is 0.1 and wherein ‘Ag’ is silver derived from silver nanoparticles. In some embodiments of the invention, the catalyst has a formula represented by Ag_{0.083}Sr_{1.0}La_{0.9}Nd_{0.7}Yb_{0.1}Oₓ, wherein the relative molar ratio ‘z’ is 0.083, the relative molar ratio ‘a’ is 1, the relative molar ratio ‘b’ is 0.9, the relative molar ratio ‘c’ is 0.7, and the relative molar ratio ‘d’ is 0.1. Further, without wishing to be bound by any specific theory and by way of this disclosure, it is believed that the synergistic combination of rare earth elements such as lanthanum, which promotes OCM catalyst activity, with rare earth element such as neodymium, which promotes C₂₊ hydrocarbon selectivity, enables the composition containing the catalyst of the present invention, to demonstrate improved catalyst activity while retaining the desired level of selectivity.

In some aspects of the invention, the invention provides a method for preparing the composition containing the catalyst of the present invention, comprising a silver promoter and a mixed metal oxide containing an alkaline earth metal element and at least two rare earth metal elements, where the method comprises: (i) forming an aqueous catalyst precursor solution comprising a silver agent and a precursor mixture comprising an alkali earth metal compound and at least two rare earth metal compounds; (ii) drying the aqueous catalyst precursor solution at a temperature of at least 90° C., alternatively at a temperature ranging from about 110° C. to about 140° C., alternatively at a temperature ranging from about 115° C. to about 130° C., and forming a dried catalyst precursor mixture; and subsequently (iii) calcining the dried catalyst precursor mixture for at least 5 hours, or alternatively for at least 6 hours, at a temperature of at least 650° C., alternatively at a temperature of at least 700° C., or alternatively at a temperature of at least 850° C., and forming the composition. In some embodiments of the invention, the method for preparing the composition comprising the catalyst of the present invention, further comprises calcining the precursor mixture and forming a calcined precursor mixture. In some embodiments of the invention, the calcined precursor mixture is mixed with the silver agent to form the aqueous catalyst precursor solution. In some embodiments of the invention, the precursor mixture comprises (i) a compound containing the alkaline earth metal (AE), (ii) a compound containing the first rare earth element (RE1), (iii) a compound containing the second rare earth metal (RE2) and optionally, (iv) a compound containing any one of, the third rare earth metal (RE3) or the redox agent. Non-limiting examples of compounds used as a precursor mixture include nitrates, carbonates, acetates, halides, oxides, hydroxides and any combinations thereof. In some preferred embodiments of the invention, the compound chosen is a nitrate salt of the alkaline earth metal (AE), a nitrate salt of the first rare earth element (RE1), a nitrate salt of the second rare earth element (RE2), a nitrate salt of the third rare earth element (RE3), and the nitrate salt of the redox agent. In some embodiments of the invention, calcination of the dried catalyst precursor mixture or of the precursor mixture can be carried out at a temperature ranging from about 700° C. to about 1,000° C., alternatively from about 750° C. to about 850° C.

The silver agent used for preparing the composition containing the catalyst of the present invention, functions as a precursor material for incorporating silver in the inventive catalyst compositions. In some embodiments of the invention, the silver agent is selected from the group consisting of silver nanoparticles, silver nanowires, silver salts and combinations thereof. In some preferred embodiments of the invention, the silver agent is a silver nanoparticle. In some embodiments of the invention, the silver nanoparticle has a particle size ranging from about m to about 500 nm, alternatively from about 10 nm to about 200 nm, alternatively from about 50 nm to about 100 nm. In some aspects of the invention, the silver nanoparticle is present in the form of an aqueous solution having a plurality of nanoparticles present at a concentration ranging from about 10,000 ppm to about 100,000 ppm, alternatively from about 20,000 ppm to about 60,000 ppm, or alternatively from about 40,000 ppm to about 55,000 ppm. For the purposes of this invention, the aqueous solution containing the plurality of silver nanoparticles, may be procured commercially from suppliers such as Sigma Aldrich. In some preferred embodiments of the invention, the aqueous solution containing silver nanoparticles is mixed with the calcined precursor mixture to form the aqueous catalyst precursor solution. In some embodiments of the invention, the silver agent is a silver salt. Non limiting examples of silver salt include silver nitrate, silver chloride, silver iodide, silver bromide. In some preferred embodiments of the invention, the silver salt is silver nitrate.

In some aspects of the invention, a composition comprising a C₂₊ hydrocarbon mixture product is formed using the composition containing the catalyst of the present invention, comprising a silver promoter and a mixed metal oxide containing an alkaline earth metal element and at least two rare earth metal elements. In some aspects of the invention, C₂₊ hydrocarbon mixture product comprises ethylene, ethane, ethyne, propene, propane, C₄-C₅ hydrocarbons, carbon dioxide, carbon monoxide and combinations thereof. In some aspects of the invention, the invention relates to a process for producing a C₂₊ hydrocarbon mixture product, using the composition containing the catalyst of the present invention, comprising a silver promoter and a mixed metal oxide containing an alkaline earth metal element and at least two rare earth metal elements. The process comprises (a) introducing a feed mixture comprising methane and oxygen in a reactor comprising the composition containing the catalyst of the present invention, (b) subjecting the feed mixture to a methane coupling reaction under conditions suitable to produce the C₂₊ hydrocarbon mixture product, and (c) recovering the C₂₊ hydrocarbon mixture product after removing unconverted methane and steam from the C₂₊ hydrocarbon mixture product. In some preferred embodiments of the invention, a portion of the C₂₊ hydrocarbon mixture product is recovered. In some aspects of the invention, unconverted methane, and steam is removed from the C₂₊ hydrocarbon mixture product. In some embodiments of the invention, the removal of unconverted methane and steam from the C₂₊ hydrocarbon mixture product is effected using a distillation column. In some embodiments of the invention, the distillation column is a cryogenic distillation column.

In some embodiments of the invention, the feed mixture comprising methane and oxygen is preheated to a feed temperature prior to introducing the feed mixture in the reactor. In some aspects of the invention, the feed mixture comprising methane and oxygen is introduced in the reactor at a feed temperature of less than 400° C. In some embodiments of the invention, the feed mixture comprising methane and oxygen is introduced at a feed temperature ranging from about 150° C. to about 380° C., alternatively from about 200° C. to about 350° C., or alternatively from about 250° C. to about 300° C. Further, as will be appreciated by one skilled in the art, and with the help of this disclosure, with a lower feed temperature to activate methane, there will be energy savings as well as eliminate the need of employing capital expensive heat exchangers for heating and activating the methane in the feed mixture. Without wishing to be bound by any specific theory, the composition containing the catalyst of the present invention, enables the feed mixture to be fed in the reactor at a temperature previously unseen for oxidative coupling of methane. In some aspects of the invention, the reactor comprises (a) an inlet for receiving a feed mixture comprising methane and oxygen, (b) a reaction zone that is configured to be in fluid communication with the inlet, wherein the reaction zone comprises a reactor tube and the composition containing the catalyst of the present invention and (c) an outlet configured to be in fluid communication with the reaction zone and configured to remove the C₂₊ hydrocarbon mixture product from the reaction zone.

The reactor can comprise an adiabatic reactor, an autothermal reactor, an isothermal reactor, a tubular reactor, a cooled tubular reactor, a continuous flow reactor, a fixed bed reactor, a fluidized bed reactor, a moving bed reactor, and the like, or combinations thereof. In some preferred embodiments of the invention, the reactor is an adiabatic reactor. In preferred aspects of the invention, a 2.3 mm ID quartz tube reactor is used for the purposes of reacting oxygen with methane under conditions sufficient to effect the oxidative coupling of methane.

In some aspects of the invention, the reactor can comprise a catalyst bed comprising the composition containing the catalyst of the present invention, capable of catalyzing the oxidative coupling of methane. In some embodiments of the invention, the ratio of methane to oxygen ranges from about 2:1 to about 15:1, alternatively from about 4:1 to about 10:1, alternatively from about 5:1 to about 8:1. Advantageously, the inventive catalyst composition of the present invention is capable of operating and retaining its activity even when subjected to high methane to oxygen ratio without deterioration of catalyst performance. In some embodiments of the invention, the pressure in the reactor is maintained at a pressure sufficient to effect oxidative coupling of methane. The pressure may be maintained at a range of about 14.7 psi (ambient atmospheric pressure) to about 500 psi, alternatively at a range of about 14.7 psi (ambient atmospheric pressure) to about 200 psi, alternatively at a range of about 14.7 psi (ambient atmospheric pressure) to about 150 psi. In some embodiments of the invention, the feed mixture is introduced into the reactor at a gas hourly space velocity (GHSV) ranging from about 500 h⁻¹ to about 1,000,000 h⁻¹, alternatively from about 1,000 h⁻¹ to about 300,000 h⁻¹, alternatively from about 5,000 h⁻¹ to about 100,000 h⁻¹, alternatively from about 10,000 h⁻¹ to about 80,000 h⁻¹, alternatively from about 20,000 h⁻¹ to about 50,000 h⁻¹.

In some aspects of the invention, the C₂₊ hydrocarbon mixture product is produced at a reactor temperature ranging from about 300° C. to about 800° C., alternatively from about 350° C. to about 720° C., alternatively from about 400° C. to about 500° C., or alternatively from about 420° C. to about 450° C. The term reactor temperature as used herein includes the reactor tube temperature at which the methane and oxygen react under conditions of oxidative coupling of methane to form C₂₊ hydrocarbon mixture product. In some aspects of the invention, the composition containing the catalyst of the present invention demonstrates high catalyst activity. One suitable metric to express catalyst activity once measured, is by reporting the temperature (T(90%)° C.) at which the 90% of the oxygen present in the feed is converted or has reacted with methane. In other words, (T(90%)° C.) value serves as a convenient proxy for ascertaining the catalyst activity of the composition containing the catalyst of the present invention. As the overall oxidative coupling reaction is exothermic in nature, lower the temperature at which 90% oxygen conversion is achieved, better is the catalyst activity. Alternatively it may be concluded that the value of (T(90%)° C.) is inversely proportional to the catalyst activity of a catalyst composition, as a result a higher value of (T(90%)° C.) indicates reduced catalyst activity while a lower value of (T(90%)° C.) is indicative of increased catalyst activity. In some embodiments of the invention, the composition has a 90% oxygen conversion temperature (T(90%)° C.) ranging from about 200° C. to about 700° C., alternatively from about 300° C. to about 600° C., alternatively from about 400° C. to about 500° C., when the composition is used in a process for producing C₂₊ hydrocarbon from methane and oxygen. The (T(90%)° C.) value can be measured by using an online Gas Chromatograph, Agilent 7890 GC, having a thermal conductivity detector (TCD) and a flame ionization detector (FID) to detect the concentration of the oxygen at the outlet of the reactor. In some preferred embodiments of the invention, the composition achieves 100% oxygen conversion at a temperature below 800° C., alternatively at a temperature below 500° C., or alternatively at a temperature below 450° C., or alternatively at a temperature below 400° C., when the composition is used in a process for producing C₂₊ hydrocarbon mixture product from methane and oxygen. For this invention, the temperature at which the OCM reaction is catalyzed with 100% oxygen conversion, is particularly significant in terms of catalyst activity, as ordinarily OCM reactions generally achieve 100% oxygen conversion at temperatures typically between 750° C. to 1050° C.

The oxygen conversion can be measured by measuring the oxygen concentration at the inlet and outlet in accordance with the equation below: (O₂ (inlet)−O₂ (outlet)/O₂ (inlet))×100, where O₂ (inlet) is the concentration of oxygen at the inlet of the reactor and O₂ (outlet) is the concentration of oxygen at the outlet. The temperature at which ninety percent (T(90%)° C.) oxygen conversion occurs may be noted using a temperature thermal couple. The thermocouple used for the purposes of this invention may be any of the commercially available thermocouple available such as thermocouples manufactured by OMEGA™. In some embodiments of invention, the composition containing the catalyst of the present invention, demonstrates sufficient methane conversion while retaining desired level of C₂₊ hydrocarbon selectivity. In some aspects of the invention, the composition containing the catalyst of the present invention has a methane conversion value ranging from about 10% to about 50%, alternatively from about 12% to about 30%, or alternatively from about 15% to about 23%. The methane conversion can be measured by using methane concentration using a gas chromatograph. The methane conversion can be determined using the equation: (CH₄ (inlet)−CH₄ (outlet)/CH₄ (inlet))×100, where CH₄ (inlet) is the concentration of methane at the inlet of the reactor and CH₄ (outlet) is the concentration of methane at the outlet.

Without wishing to be bound by any specific theory, it is believed that the presence of silver promoter in specific combination with the mixed metal oxide containing an alkaline earth metal element and at least two rare earth metal elements, for the composition containing the catalyst of the present invention, imparts high catalyst activity, which enables the methane coupling reaction to take place at a relatively lower temperature. In some embodiments of the invention, the composition has a 90% oxygen conversion temperature (T(90%)° C.) ranging from about 2% to about 80%, alternatively from about 5% to about 50%, or alternatively from about 10% to about 30%, lower than 90% oxygen conversion temperature (T(90%)° C.) of a silver-free catalyst composition having the formula (AEₐRE1_{b}RE2_{c}AT_{d}Oₓ) wherein (i) ‘AE’ represents an alkaline earth metal; (ii) ‘RE1’ represents a first rare earth element; (iii) ‘RE2’ represents a second rare earth element; and (iv) ‘AT’ represents a third rare earth element ‘RE3’, or a redox agent selected from antimony, tin, nickel, chromium, molybdenum, tungsten; wherein, ‘a’, ‘b’, ‘c’, and ‘d’ represents relative molar ratio; wherein ‘a’ is 1; ‘b’ ranges from about 0.1 to about 10; ‘c’ ranges from about 0.01 to about 10; ‘d’ ranges from 0 to about 10; ‘x’ balances the oxidation state; wherein, the first rare earth element, the second rare earth element and the third rare earth element, are different. Further, as will be appreciated by one of skill in the art, and with the help of this disclosure, a decrease in the temperature required to achieve a 90% oxygen conversion leads to a decrease in the overall OCM reaction temperature, which further leads to a decreased catalyst bed temperature and thereby reduces chances of catalyst degradation, and formation of deep oxidation byproducts.

As may be appreciated by a person skilled in the art, the selectivity and activity properties of a catalyst are generally of opposing attributes. In some aspects of the invention, the inventors surprisingly found that the composition containing the catalyst comprising silver promoter and a mixed metal oxide containing an alkaline earth metal and at least two rare earth metals, demonstrates excellent catalyst activity even while retaining sufficient selectivity as required for industrial scale use of oxidative coupling of methane. In some aspects of the invention, the composition has a C₂₊ hydrocarbon selectivity ranging from about 70% to about 88%, alternatively from about 75% to about 85%, or alternatively from about 78% to about 82%, of total product formed when the composition is used in a process for producing C₂₊ hydrocarbon from methane and oxygen. The selectivity for C₂₊ hydrocarbon can be measured at a temperature ranging from about 300° C. to about 800° C. In a preferred embodiment of the invention, the selectivity for C₂₊ hydrocarbon is measured from 500° C. to about 800° C. For the purposes of this invention, the maximum selectivity for C₂₊ hydrocarbon obtained is indicative of the selectivity property of the catalyst composition. The selectivity property exhibited by the inventive catalyst composition, results in lowering of the overall heat produced during the coupling reaction, improving catalyst performance and aiding in controlling reactor operations.

Without wishing to be bound by any specific theory and as demonstrated by way of specific examples provided in this disclosure, it is suspected that the presence of silver promoter in specific combination with the mixed metal oxide containing an alkaline earth metal and at least two rare earth metals as contemplated for the composition containing the catalyst of the present invention, enables the composition to retain sufficient selectivity even while demonstrating high catalyst activity. In some embodiments of the invention, the composition has a C₂₊ hydrocarbon selectivity ranging from about 98% to about 105% of C₂₊ hydrocarbon selectivity of the silver-free catalyst composition having the formula (AEₐRE1_{b}RE2_{c}AT_{d}Oₓ) where (i) ‘AE’ represents an alkaline earth metal; (ii) ‘RE1’ represents a first rare earth element; (iii) ‘RE2’ represents a second rare earth element; and (iv) ‘AT’ represents a third rare earth element ‘RE3’, or a redox agent selected from antimony, tin, nickel, chromium, molybdenum, tungsten; wherein, ‘a’, ‘b’, ‘c’, and ‘d’ represents relative molar ratio; wherein ‘a’ is 1; ‘b’ ranges from 0.1 to 10; ‘c’ ranges from about 0.01 to about 10; ‘d’ ranges from 0 to about 10; ‘x’ balances the oxidation state; wherein, the first rare earth element, the second rare earth element and the third rare earth element, are different. Without wishing to be limited by theory, and as evidenced by way of the examples, the promotion effect of silver (Ag) on OCM catalysts is to improve (e.g., increase) the rate of the re-oxidation step of the catalyst and the formation of methyl radical, which is believed to be the rate determining step for the OCM reaction for all OCM catalysts. Further, in the present invention, the synergistic incorporation of silver promoter with a mixed metal oxide containing an alkaline earth metal element and at least two rare earth metal elements, creates a catalyst composition having improved catalyst activity and C₂₊ hydrocarbon selectivity even when compared to previously known silver promoted silver OCM catalysts.

Accordingly, the invention includes various embodiments related to catalyst compositions that exhibit one or more benefits of having high catalytic activity even when subjected to a relatively low OCM temperature while retaining sufficient catalyst selectivity for producing C₂₊ hydrocarbon products. Advantageously, the invention now enables artisans to formulate catalyst compositions in such a manner so as to enable the catalysis of the coupling reaction between methane and oxygen even at low reactor temperature, thereby preventing catalyst decomposition and preventing the formation of undesirable oxidation byproducts. Further, with the inventive catalyst having high reactivity even at low temperature, allows low catalyst loading and eliminates the need for heat exchangers to effect the OCM reaction, leading to capital and operational cost savings.

Specific examples demonstrating some of the embodiments of the invention are included below. The examples are for illustrative purposes only and are not intended to limit the invention. It should be understood that the embodiments and the aspects disclosed herein are not mutually exclusive and such aspects and embodiments can be combined in any way. Those of ordinary skill in the art will readily recognize parameters that can be changed or modified to yield essentially the same results.

### EXAMPLES

### Example 1

Catalyst Composition Having the Formula (Ag_{0.11}Sr₁La_{0.9}Yb_{0.1}Oₓ) Formed by Using Silver Salt Precursor.

Purpose:

Example 1 demonstrates the preparation and use of a composition comprising a silver promoted catalyst, having the formula (Ag_{0.11}Sr₁La_{0.9}Yb_{0.1}Oₓ). The incorporation of silver in the catalyst composition is achieved by using a silver salt as the silver agent functioning as the precursor material. The composition is used for the production of C₂₊ hydrocarbon mixture product at high catalyst activity determined by way of (T(90%)° C.) measurement while retaining sufficient selectivity towards C₂₊ hydrocarbon mixture product. The performance of the inventive composition of Example 1 is further compared and contrasted with a control containing a catalyst composition having an identical composition as that of Example 1 but without the presence of silver.

Materials:

The following materials are procured and used for the synthesis of the composition.

TABLE 1
Inventive catalyst composition (Ag_{0.11}Sr_{1.0}La_{0.9}Yb_{0.1}Oₓ)

| First catalyst component: | | Relative | Precursor | |
|---|---|---|---|---|
| Ag_{z}AEₐRE1_{b}RE2_{c}RE3_{d}Oₓ | Element used | molar ratio | Material | Supplier |
| Ag | Silver (Ag) | z = 0.11 | AgNO₃ | Sigma-Aldrich |
| AE | Strontium (Sr) | a = 1.0 | Strontium | Sigma-Aldrich |
| | | | Nitrate: Sr(NO₃)₂ | |
| RE1 | Lanthanum (La) | b = 0.9 | Lanthanum | Sigma-Aldrich |
| | | | Nitrate | |
| | | | (La(NO₃)₃•6H₂O) | |
| RE2 | Ytterbium (Yb) | c = 0.1 | Ytterbium | Sigma-Aldrich |
| | | | Nitrate: | |
| | | | Yb(NO₃)₃•5H₂O | |
| RE3 | NA | d = 0 | NA | NA |

Method for Preparing the Composition Containing the Inventive Catalyst of Example 1 (Ag_{0.11}Sr₁La_{0.9}Yb_{0.1}Oₓ):

The composition was prepared by a method of (i) forming an aqueous catalyst precursor solution comprising a silver agent and a precursor mixture comprising an alkali earth metal compound and at least two rare earth metal compound, which was followed by (ii) drying the aqueous catalyst precursor solution at a temperature of at least 90° C. and forming a dried catalyst precursor mixture; and (iii) calcining the dried catalyst precursor mixture for at least 5 hours at a temperature of at least 650° C. and subsequently forming the composition containing the catalyst of the present invention. More specifically, the method included the step of forming an aqueous catalyst precursor solution containing in about 50 ml of water about 0.76 g of silver nitrate salt (Ag(NO₃)) as the silver agent, and the precursor mixture containing 8.47 g of strontium nitrate (Sr(NO₃)₂), 15.59 g of lanthanum nitrate (La(NO₃)₃.6H₂O) and 1.8 g of Yb(NO₃)₃.5H₂O. The aqueous catalyst precursor solution was then dried at a temperature of 125° C. overnight, to form the dried catalyst precursor mixture. The dried catalyst precursor mixture was then calcined at 900° C. for 6 hours to obtain the inventive composition of Example 1. A reference catalyst composition (Reference 1) to be used as a control to the inventive composition of Example 1 was also prepared as described below:

Method for preparing the Reference 1, a silver free catalyst composition to be used as a control for the inventive composition of Example 1, with the control having the composition (Sr_{1.0}La_{0.9}Yb_{0.1}Oₓ):

The following steps were followed for the synthesis of Reference 1 composition: 4.23 g of (Sr(NO₃)₂), 7.82 g of (La(NO₃)₃.6H₂O) and 0.9 g of (Yb(NO₃)₃.5H₂O) were mixed and dissolved in 25 ml water to form a solution. The solution was subsequently dried overnight at a temperature of 125° C. followed by calcination at 900° C. for 6 hours to obtain the control composition Reference 1.

Process for Producing C₂₊ Hydrocarbon Mixture Product Using the Composition of Example 1:

The composition obtained from the practice of Example 1, was thereafter used for producing C₂₊ hydrocarbon mixture product using the process comprising (a) introducing a feed mixture comprising methane and oxygen in a reactor containing the inventive composition of Example 1; (b) subjecting the feed mixture to a methane coupling reaction under conditions suitable to produce the C₂₊ hydrocarbon mixture product; and (c) recovering the C₂₊ hydrocarbon mixture product after removing unconverted methane and steam from the C₂₊ hydrocarbon mixture product. More particularly, the composition containing the catalyst obtained from Example 1, was placed in a 2.3 mm ID quartz tube, and was contacted with a feed mixture containing methane and oxygen. The ratio of methane to oxygen was adjusted to a ratio of 7.4:1 and the feed mixture flow rate was adjusted from 40 sccm. The catalyst loading in the reactor was 20 mg. The reactors were operated under ambient pressure and catalyst performance under different reactor temperatures was accordingly determined. The C₂₊ hydrocarbon mixture product so obtained was analyzed using online Gas Chromatograph, Agilent 7890 GC, having a thermal conductivity detector (TCD) and a flame ionization detector (FID). The C2+ hydrocarbon selectivity was measured at different temperatures during the course of the OCM reaction and the maximum value was noted.

The operating parameters for producing the C₂₊ hydrocarbon mixture product is as given below:

TABLE 3
Operating Parameter used for producing
C₂₊ hydrocarbon mixture product

| Pressure inside reactor (psi) | Gas Hourly Space Velocity (GHSV) (hr⁻¹) |
|---|---|
| Ambient pressure, (14.7) | 115,589 |

The catalyst composition of Reference 1 was subjected to the same reaction condition and process steps as that of the composition of Example 1. The performance of the inventive catalyst composition of Example 1 was subsequently compared with the performance of Reference 1.

Results:

The catalyst performance obtained using the catalyst composition of Example 1 and catalyst performance obtained from the use of the catalyst composition of Reference 1, are tabulated below.

TABLE 4
Catalyst selectivity/Activity

| | | Maximum C₂₊ |
|---|---|---|
| | Catalyst Activity - T(90%)° C. - | hydrocarbon |
| | temperature at which 90% | product |
| | oxygen conversion is achieved | selectivity |
| Inventive composition | 675° C. | 79.8 |
| Example 1 | | |
| (Control) Reference 1 | 700° C. | 80.3 |

The results from Table 4 indicate that inventive catalyst composition of Example 1, has a 90% oxygen conversion temperature (T(90%)° C.) which is 3.5% lower than the silver free catalyst composition of Reference 1. In other words, the inventive composition obtained from the practice of Example 1, shows increased catalyst activity by demonstrating a lower temperature at which 90% oxygen conversion is achieved (675° C.), when compared with the temperature at which 90% oxygen conversion is achieved for Reference 1 (700° C.). Further, contrary to expectation that the increase in catalyst activity would adversely affect the C₂₊ hydrocarbon selectivity performance of the inventive composition, the inventors surprisingly found that the selectivity of the inventive catalyst composition of Example 1 is almost similar to that of Reference 1 and in particular the inventive composition has a C₂₊ hydrocarbon selectivity of about 99.3% of the C₂₊ hydrocarbon selectivity of Reference 1. Thus it may be concluded from the results of Example 1, that the inventive catalyst composition of Example 1 demonstrates previously unseen benefits of a catalyst composition for oxidative coupling of methane, having high catalyst activity even when subjected to a relatively low reactor temperature, without affecting the performance of C₂₊ hydrocarbon selectivity of the catalyst.

### Example 2

Catalyst Composition Having the Formula (Ag_{0.083}Sr_{1.0}La_{0.9}Nd_{0.7}Yb_{0.1}Oₓ) Formed by Using Silver Salt Precursor.

Purpose:

Example 2 demonstrates the preparation and use of a composition comprising a silver promoted catalyst, having the formula (Ag_{0.083}Sr_{1.0}La_{0.9}Nd_{0.7}Yb_{0.1}Oₓ). The incorporation of silver in the catalyst composition is achieved by using a silver salt as the silver agent functioning as the precursor material. The composition is used for the production of C₂₊ hydrocarbon mixture product at high catalyst activity determined by way of (T(90%)° C.) measurement while retaining or improving selectivity towards C₂₊ hydrocarbon mixture product. The inventive composition of Example 2 is further compared with a control containing a catalyst composition having an identical composition as that of Example 2 but without the presence of silver.

Materials:

The following materials are procured and used for the synthesis of the composition.

TABLE 5
Inventive catalyst composition (Ag_{0.083}Sr_{1.0}La_{0.9}Nd_{0.7}Yb_{0.1}Oₓ)

| First catalyst component: | | Relative | Precursor | |
|---|---|---|---|---|
| Ag_{z}AEₐRE1_{b}RE2_{c}RE3_{d}Oₓ | Element used | molar ratio | Material | Supplier |
| Ag | Silver (Ag) | z = 0.083 | AgNO₃ | Sigma-Aldrich |
| AE | Strontium (Sr) | a = 1.0 | Strontium | Sigma-Aldrich |
| | | | Nitrate: Sr(NO₃)₂ | |
| RE1 | Lanthanum (La) | b = 0.9 | Lanthanum | Sigma-Aldrich |
| | | | Nitrate | |
| | | | (La(NO₃)₃•6H₂O) | |
| RE2 | Neodymium (Nd) | c = 0.7 | Neodymium | Sigma-Aldrich |
| | | | Nitrate: | |
| | | | Nd(NO₃)₃•6H₂O | |
| RE3 | Ytterbium (Yb) | d = 0.1 | Ytterbium | Sigma-Aldrich |
| | | | Nitrate: | |
| | | | Yb(NO₃)₃•5H₂O | |

Method for Preparing the Composition Containing the Inventive Catalyst of Example 2 (Ag_{0.083}Sr_{1.0}La_{0.9}Nd_{0.7}Yb_{0.1}Oₓ):

The inventive composition was prepared by a method similar to what was described under Example 1. The method of preparation included the steps of forming an aqueous catalyst precursor solution containing in about 25 ml of water about 0.57 g of silver nitrate salt (Ag(NO₃)) as the silver agent, and the precursor mixture containing 8.47 g of strontium nitrate (Sr(NO₃)₂), 15.59 g of lanthanum nitrate (La(NO₃)₃.6H₂O), 12.28 g of neodymium nitrate (Nd(NO₃)₃.6H₂O) and 1.8 g of Yb(NO₃)₃.5H₂O. The aqueous catalyst precursor solution was then dried overnight at a temperature of 125° C. overnight to form the dried catalyst precursor mixture. The dried catalyst precursor mixture was then calcined at 900° C. for 6 hours to obtain the inventive composition of Example 2. A reference catalyst composition (Reference 2) to be used as a control to the inventive composition of Example 2 was also prepared as described below:

Method for Preparing the Reference 2, a Silver Free Catalyst Composition to be Used as a Control for the Inventive Composition of Example 2 with the Control Having the Composition (Sr_{1.0}La_{0.9}Yb_{0.1}Oₓ):

The following steps were followed for the synthesis of Reference 2 composition: 4.23 g of strontium nitrate (Sr(NO₃)₂), 7.82 g of lanthanum nitrate (La(NO₃)₃.6H₂O), 6.14 g of neodymium nitrate (Nd(NO₃)₃.6H₂O) and 0.9 g of Yb(NO₃)₃.5H₂O, were mixed and dissolved in 25 ml water to form a solution. The solution was subsequently dried overnight at a temperature of 125° C. followed by calcination at 900° C. for 6 hours to obtain the control composition Reference 2 (Sr_{1.0}La_{0.9}Yb_{0.1}Oₓ).

Process for Producing C₂₊ Hydrocarbon Mixture Product Using the Composition of Example 2:

The process practiced for Example 2, was identical to what was practiced under Example 1, except that in the present instant the inventive catalyst composition obtained from Example 2 was used. The operating parameters for producing the C₂₊ hydrocarbon mixture product was kept identical as described under Example 1. The catalyst composition of Reference 2 (Sr_{1.0}La_{0.9}Yb_{0.1}Oₓ), was subjected to the same reaction condition and process steps as that of the composition of Example 2 and the performance of the catalyst composition is recorded as described below.

Results:

The catalyst performance obtained using the catalyst composition of Example 2 and catalyst performance obtained from the use of the catalyst composition of Reference 2, are tabulated below using the measurement technique described in Example 1.

TABLE 6
Catalyst selectivity/Activity

| | | Maximum C₂₊ |
|---|---|---|
| | Catalyst Activity - T(90%)° C. - | hydrocarbon |
| | temperature at which 90% | product |
| | oxygen conversion is achieved | selectivity |
| Inventive composition | 575° C. | 80.3 |
| Example 2 | | |
| (Control) Reference 2 | 625° C. | 79.5 |

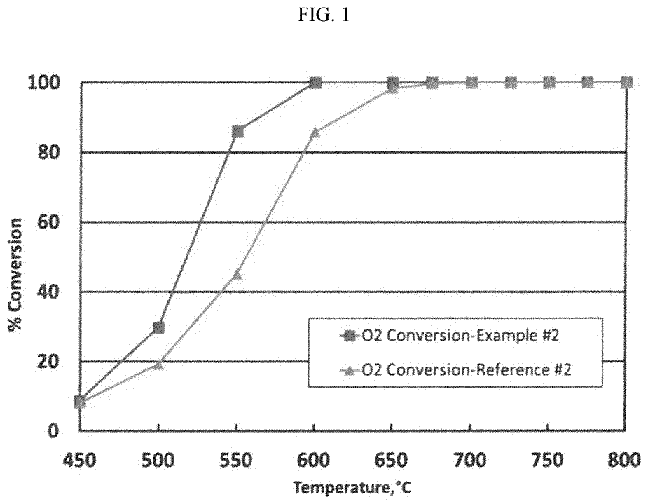

FIG. 1, provides a graphical overview of the oxygen conversion measured at different reactor temperature. As is evident from FIG. 1, the inventive catalyst composition of Example 2, demonstrates higher oxygen conversion at a lower temperature as compared to the control Reference 2, thereby indicating improved catalyst activity over that of Reference 2. The results from Table 6 indicate that inventive catalyst composition of Example 2, has a 90% oxygen conversion temperature (T(90%)° C.) which is about 8% lower than the silver free catalyst composition of Reference 2. As observed with Example 1, the inventive composition obtained from the practice of Example 2, shows increased catalyst activity by demonstrating a significantly lower temperature at which 90% oxygen conversion is achieved (575° C.) when compared with the temperature at which 90% oxygen conversion is achieved for Reference 2 composition (625° C.). Further, contrary to expectation that the increase in catalyst activity would adversely affect the C2+ hydrocarbon selectivity performance of the inventive catalyst composition, the inventors surprisingly found that the selectivity of the inventive catalyst composition of Example 2, marginally increased from that of Reference 2. Particularly the inventive composition has a C2+ hydrocarbon selectivity of about 101% of the C2+ hydrocarbon selectivity of Reference 2. The results from the practice of Example 2, were particularly significant as catalyst activity and selectivity tend to be of opposing attributes. Further, as with Example 1, the advantage of keeping the feed temperature low, enables increased operational efficiency with low capital and operational expenditure and reduced levels of deep oxidation byproducts. Thus it may be concluded, that the inventive catalyst composition of Example 2, demonstrates previously unseen benefits of a catalyst composition for oxidative coupling of methane, having improved catalyst activity even when operated at low reactor temperature, as well as marginal increase in the C2+ hydrocarbon selectivity performance.

### Example 3

Catalyst Composition Having the Formula (Ag_{0.17}Sr_{1.0}La_{0.9}Nd_{0.7}Yb_{0.1}Oₓ) Formed by Using Silver Salt Precursor

Purpose:

Example 3 demonstrates the preparation and use of a composition comprising a silver promoted catalyst, having the formula (Ag_{0.17}Sr_{1.0}La_{0.9}Nd_{0.7}Yb_{0.1}Oₓ). The incorporation of silver in the catalyst composition is achieved by using a silver salt as the silver agent, functioning as the precursor material. The composition is used for the production of C₂₊ hydrocarbon mixture product at high catalyst activity determined by way of (T(90%)° C.) measurement while retaining sufficient selectivity towards C₂₊ hydrocarbon mixture product. The inventive composition of Example 3 is further compared and contrasted with a control containing a catalyst composition having an identical composition as that of Example 3 but without the presence of silver.

Materials:

The material used were same as reported under Example 2, except that silver was used at a different relative molar ratio.

Method for Preparing the Composition Containing the Inventive Catalyst of Example 3 (Ag_{0.17}Sr_{1.0}La_{0.9}Nd_{0.7}Yb_{0.1}Oₓ):

The inventive composition was prepared by a method similar to what was described under Example 1 and Example 2. The method of preparation included the steps of forming an aqueous catalyst precursor solution containing about 25 ml of water, about 1.16 g of silver nitrate salt (Ag(NO₃)) as the silver agent, and the precursor mixture containing 8.47 g of strontium nitrate (Sr(NO₃)₂), 15.59 g of lanthanum nitrate (La(NO₃)₃.6H₂O), 12.28 g of neodymium nitrate (Nd(NO₃)₃.6H₂O) and 1.8 g of Yb(NO₃)₃.5H₂O. The aqueous catalyst precursor solution was then dried overnight at a temperature of 125° C. to form the dried catalyst precursor mixture. The dried catalyst precursor mixture was then calcined at 900° C. for 6 hours to obtain the inventive composition of Example 3. Reference 2 composition (Sr_{1.0}La_{0.9}Nd_{0.7}Yb_{0.1}Oₓ), prepared for evaluating the performance of Example 2, was used as a control to the inventive composition of Example 3.

Process for Producing C₂₊ Hydrocarbon Mixture Product Using the Composition of Example 3:

The process practiced was identical to what was practiced under Example 1 and Example 2, except that in the present instant the inventive catalyst composition obtained from Example 3 was used. The operating parameters for producing the C₂₊ hydrocarbon mixture product was kept identical as described under Example 1. The catalyst composition of Reference 2 (Sr_{1.0}La_{0.9}Nd_{0.7}Yb_{0.1}Oₓ), was subjected to the same reaction condition and process steps as that of the inventive composition of Example 3, and the performance of the catalyst composition was recorded as described below.

Results:

The catalyst performance obtained using the catalyst composition of Example 3 and catalyst performance obtained from the use of the catalyst composition of Reference 2, are tabulated below using the measurement technique described in Example 1 and Example 2.

TABLE 7
Catalyst selectivity/Activity

| | | Maximum C₂₊ |
|---|---|---|
| | Catalyst Activity - T(90%)° C. - | hydrocarbon |
| | temperature at which 90% | product |
| | oxygen conversion is achieved | selectivity |
| Inventive composition | 575° C. | 79.0 |
| Example 3 | | |
| (Control) Reference 2 | 625° C. | 79.5 |

The results from Table 7 indicate that inventive catalyst composition of Example 3, has a 90% oxygen conversion temperature (T(90%)° C.) which is about 8% lower than the silver free catalyst composition of Reference 2. As observed, the inventive composition obtained from the practice of Example 3, shows increased catalyst activity by demonstrating a significantly lower temperature at which 90% oxygen conversion is achieved (575° C.) when compared with the temperature at which 90% oxygen conversion is achieved for Reference 2 (625° C.). As with Example 1, the C₂₊ hydrocarbon selectivity retained similar performance levels with that of the control Reference 2 indicating that the increase in catalyst activity did not adversely impact the catalyst selectivity property. In particular the inventive composition has a C₂₊ hydrocarbon selectivity of about 99.3% of the C₂₊ hydrocarbon selectivity of Reference 2. The results were particularly significant as catalyst activity and selectivity tend to be of opposing attributes. Thus it may be concluded from the results of Example 3, that the inventive catalyst composition of Example 3 demonstrates previously unseen benefits of a catalyst composition for oxidative coupling of methane, having improved catalyst activity without impacting the C₂₊ hydrocarbon selectivity.

### Example 4

Catalyst composition having the formula (Ag_{0.046}Sr_{1.0}La_{0.9}Nd_{0.7}Yb_{0.1}Oₓ) formed by using silver nanoparticles as precursor

Purpose:

Example 4 demonstrates the preparation and use of a composition comprising a silver promoted catalyst, having the formula (Ag_{0.046}Sr_{1.0}La_{0.9}Nd_{0.7}Yb_{0.1}Oₓ). The incorporation of silver in the catalyst composition is achieved by using a silver nanoparticles as the silver agent, functioning as the precursor material. The composition is used for the production of C₂₊ hydrocarbon mixture product at high catalyst activity determined by way of (T(90%)° C.) measurement while retaining sufficient selectivity towards C₂₊ hydrocarbon mixture product. The inventive composition of Example 4 is further compared with a control containing a catalyst composition having an identical composition as that of Example 4 but without the presence of silver.

Materials:

The material used were same as reported under Example 2, except that silver nanoparticles were used. The silver nanoparticle was obtained from Sigma Aldrich, and was present in the form of an aqueous solution having a plurality of nanoparticles present at a concentration of 50,000 ppm.

Method for Preparing the Composition Containing the Inventive Catalyst of Example 4 (Ag_{0.046}Sr_{1.0}La_{0.9}Nd_{0.7}Yb_{0.1}Oₓ):

The inventive composition was prepared by a method of (i) forming an aqueous catalyst precursor solution comprising silver nanoparticles as the silver agent and a calcined precursor mixture comprising an alkaline earth metal element and at least two rare earth metal elements, which was followed by (ii) drying the aqueous catalyst precursor solution at a temperature of at least 90° C. and forming a dried catalyst precursor mixture; and (iii) calcining the dried catalyst precursor mixture for at least 5 hours at a temperature of at least 650° C. and subsequently forming the composition containing the catalyst of the present invention. For the purpose of Example 4, the calcined precursor mixture used was Reference 2 composition (Sr_{1.0}La_{0.9}Nd_{0.7}Yb_{0.1}Oₓ) prepared as described in Example 2 and Example 3. The method of preparation included the steps of adding dropwise 0.25 ml of 50,000 ppm of silver nanoparticles to 0.99 g of calcined precursor mixture comprising Reference 2 composition (Sr_{1.0}La_{0.9}Nd_{0.7}Yb_{0.1}Oₓ), and followed by uniform mixing to form the aqueous catalyst precursor solution. Subsequently, the aqueous catalyst precursor solution was dried at 125° C. to form the dried catalyst precursor mixture. The dried catalyst precursor mixture was then calcined at 900° C. for 6 hours to obtain the inventive composition of Example 4. Reference 2 composition (Sr_{1.0}La_{0.9}Nd_{0.7}Yb_{0.1}Oₓ), was also used as a control to the inventive composition of Example 4.

Process for Producing C₂₊ Hydrocarbon Mixture Product Using the Composition of Example 4:

The process practiced was identical to what was practiced under Example 1 and Example 2, except that in the present instant the inventive catalyst composition obtained from Example 4 was used. The operating parameters for producing the C₂₊ hydrocarbon mixture product was kept identical as described under Example 1. The catalyst composition of Reference 2 (Sr_{1.0}La_{0.9}Nd_{0.7}Yb_{0.1}Oₓ), was subjected to the same reaction condition and process steps as that of the inventive composition of Example 4, and the performance of the catalyst composition was recorded as described below.

Results:

The catalyst performance obtained using the catalyst composition of Example 4 and catalyst performance obtained from the use of the catalyst composition of Reference 2 (Sr_{1.0}La_{0.9}Nd_{0.7}Yb_{0.1}Oₓ), are tabulated below using the measurement technique described in Example 1 and Example 2.

TABLE 8
Catalyst selectivity/Activity

| | | Maximum C₂₊ |
|---|---|---|
| | Catalyst Activity - T(90%)° C. - | hydrocarbon |
| | temperature at which 90% | product |
| | oxygen conversion is achieved | selectivity |
| Inventive composition | <450° C. | 79.7 |
| Example 4 | | |
| (Control) Reference 2 | 625° C. | 79.5 |

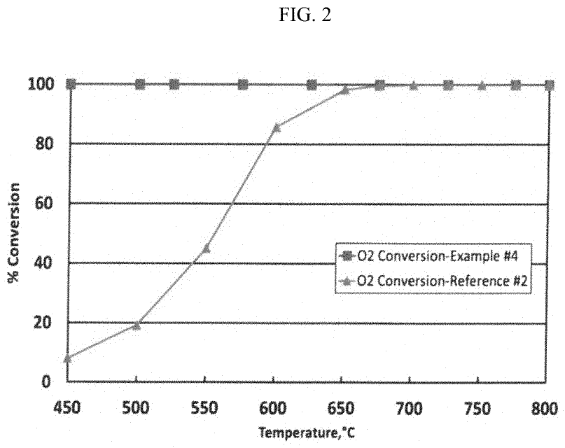

The results from Table 8 indicate that inventive catalyst composition of Example 4, has a 90% oxygen conversion temperature (T(90%)° C.), which is at least 28% lower than the silver free catalyst composition of Reference 2 (Sr_{1.0}La_{0.9}Nd_{0.7}Yb_{0.1}Oₓ). In particular, FIG. 2, illustrates the oxygen conversion measured at various reactor temperature. It was observed that, at the first point of measurement of oxygen conversion at 450° C., 100% of the oxygen present in the feed mixture was already consumed for the OCM reaction, indicating that the catalyst composition prepared under Example 4 has extremely high catalyst activity. Thus it is evident, that the temperature at which 90% oxygen conversion would have been achieved, would be much lower than 450° C. A complete conversion of oxygen at 450° C. is particularly remarkable, as it allows the OCM reaction to be conducted at a much lower temperature than what has been reported in published technical and scientific journals. Further, contrary to expectation that such an increase in catalyst activity would severely impact the C₂₊ hydrocarbon selectivity performance, the inventors surprisingly found that the selectivity of the inventive catalyst composition of Example 4, marginally increased from that of the control Reference 2. Specifically, the inventive composition has a C₂₊ hydrocarbon selectivity which is about 100.2% of the C₂₊ hydrocarbon selectivity of Reference 2. It may be further inferred that the feed temperature at which the feed is introduced in the reactor will be significantly lower than 450° C. The improved catalyst reactivity at low temperature is particularly beneficial, as heat exchangers, which have high capital and operational expenditure are not required for activating the feed prior to coupling. By maintaining a check on the feed temperature, the overall capital and operational expenditure is made viable while mitigating risk of forming deep oxidation products of carbon monoxide and carbon dioxide

Thus it may be concluded from the results of Example 4, that the inventive catalyst composition of Example 4, demonstrates previously unseen benefits of a catalyst composition for oxidative coupling of methane, having significant improvement in catalyst activity without affecting C₂₊ hydrocarbon selectivity performance, allowing OCM reactions to be effected at a significantly lower reactor temperature than what is ordinarily practiced.

### Example 5

Catalyst Composition Having the Formula (Ag_{0.091}Sr_{1.0}La_{0.9}Nd_{0.7}Yb_{0.1}Oₓ) Formed by Using Silver Nanoparticles as Precursor

Purpose:

Example 5 demonstrates the preparation and use of a composition comprising a silver promoted catalyst, having the formula (Ag_{0.091}Sr_{1.0}La_{0.9}Nd_{0.7}Yb_{0.1}Oₓ). The incorporation of silver in the catalyst composition is achieved by using a silver nanoparticles as the silver agent which functions as the precursor material. The composition is used for the production of C₂₊ hydrocarbon mixture product at high catalyst activity determined by way of (T(90%)° C.) measurement while retaining sufficient selectivity towards C₂₊ hydrocarbon mixture product. The inventive composition of Example 5 is further compared and contrasted with a control containing a catalyst composition having an identical composition as that of Example 5 but without the presence of silver.

Materials:

The material used were identical as reported under Example 4.

Method for preparing the composition containing the inventive catalyst of Example 5 (Ag_{0.091}Sr_{1.0}La_{0.9}Nd_{0.7}Yb_{0.1}Oₓ): For the purpose of Example 5, the method for preparing the inventive catalyst composition was identical as described under Example 4 except that 0.5 ml of 50,000 ppm of silver nanoparticles was added to 0.99 g of calcined precursor mixture comprising the Reference 2 composition (Sr_{1.0}La_{0.9}Nd_{0.7}Yb_{0.1}Oₓ). Reference 2 composition (Sr_{1.0}La_{0.9}Nd_{0.7}Yb_{0.1}Oₓ), was also used as a control to the inventive composition of Example 5.

Process for Producing C₂₊ Hydrocarbon Mixture Product Using the Composition of Example 5:

The process practiced was identical to what was practiced under previous examples except that the inventive composition of Example 4 was used. The operating parameters for producing the C₂₊ hydrocarbon mixture product was kept identical as described under Example 1 and that of any previous examples. The catalyst composition of Reference 2 (Sr_{1.0}La_{0.9}Nd_{0.7}Yb_{0.1}Oₓ), was subjected to the same reaction condition and process steps as that of the inventive composition of Example 5, and the performance of the catalyst composition was recorded as described below.

Results:

The catalyst performance obtained using the catalyst composition of Example 5 and catalyst performance obtained from the use of the catalyst composition of Reference 2 (Sr_{1.0}La_{0.9}Nd_{0.7}Yb_{0.1}Oₓ), are tabulated below using the measurement technique described in Example 1 and Example 2.

TABLE 9
Catalyst selectivity/Activity

| | | C₂₊ |
|---|---|---|
| | Catalyst Activity - T(90%)° C. - | hydrocarbon |
| | temperature at which 90% | product |
| | oxygen conversion is achieved | selectivity |
| Inventive composition | <450° C. | 79.4 |
| Example 5 | | |
| (Control) Reference 2 | 625° C. | 79.5 |

The results from Table 9 indicate that inventive catalyst composition of Example 5, has a 90% oxygen conversion temperature (T(90%)° C.) which is at least 28% lower than the silver free catalyst composition of Reference 2 (Sr_{1.0}La_{0.9}Nd_{0.7}Yb_{0.1}Oₓ). As was reported in Example 4, it was observed that the inventive catalyst composition of Example 5 achieved 100% oxygen conversion at a temperature of 450° C., indicating a significant improvement in catalyst activity over that of the control. Further, the C₂₊ hydrocarbon selectivity performance of the inventive catalyst composition, was retained despite such remarkable increase in catalyst activity. Thus it may be concluded from the results of Example 5, that the inventive catalyst composition of Example 5, demonstrates previously unseen benefits of a catalyst composition for oxidative coupling of methane, having significant improvement in catalyst activity without adversely affecting C₂₊ hydrocarbon selectivity performance.

### Example 6 (Comparative)

Catalyst composition having the formula 1% Ag—Mn—Na₂WO₄ (US20170014807)

Purpose:

Example 6 is used as a comparative example to compare the performance of a silver promoted catalyst composition reported in the published US patent application US20170014807 having the formula Ag—Mn—Na₂WO₄, with the inventive catalyst composition of Example 2 of the present invention. The intention of using this comparative example is to demonstrate that the inventive silver promoted composition of the present invention demonstrates improved selectivity and catalytic activity compared to previously disclosed silver promoted oxidative coupling of methane.

Method of Preparing the Manganese Promoted Second Catalyst Component:

The method of preparing the catalyst Ag—Mn—Na₂WO₄ has been described in detail in US US20170014807. The 1.0% Ag—Mn—Na2WO4/SiO2 catalyst (catalyst #6) was prepared as follows. Silica gel (18.6 g. Davisil® Grade 646) was used after drying overnight. Mn(NO₃)₂.4H₂O (1.73 g) was dissolved in deionized water (18.6 mL), and then added dropwise onto the silica gel and the material obtained was dried at overnight 125° C. Ag(NO₃) (0.32 g) was dissolved in deionized water (18.6 mL), and the solution obtained was added dropwise onto the dried manganese silica gel and the material obtained was dried at 125° C. overnight. Na₂WO₄.4H₂O (1.13 g) was dissolved in deionized water (18.6 mL), and the solution obtained was added onto the dried manganese silica material above. The resultant material obtained was dried overnight at 125° C. and calcined at 800° C. for 6 hours under airflow to produce catalyst composition of comparative Example 6.

Process for Producing C₂₊ Hydrocarbon Mixture Product Using the Composition of Example 6:

The process practiced was identical to what was practiced under any of the inventive examples from Example 1-5, except that for the purpose of this comparative example, catalyst composition of Example 6 was used and the catalyst loading was much higher. The operating parameters for producing the C₂₊ hydrocarbon mixture product was kept identical as described under Example 1 or Example 2 except that the catalyst loading as used for the comparative Example 6 was 100 mg instead of 20 mg used for the inventive examples.

Result:

The catalyst performance obtained using the catalyst composition of Example 6 (1.0% Ag—Mn—Na₂WO4/SiO₂) and catalyst performance obtained from the use of the inventive catalyst composition of Example 2 (Ag_{0.083}Sr_{1.0}La_{0.9}Nd_{0.7}Yb_{0.1}Oₓ), are tabulated below using the identical measurement technique described in Example 1 and Example 2.

TABLE 10
Catalyst selectivity/Activity

| | | Maximum C₂₊ |
|---|---|---|
| | Catalyst Activity - T(90%)° C. - | hydrocarbon |
| | temperature at which 90% | product |
| | oxygen conversion is achieved | selectivity |
| Inventive composition | 575° C. | 80.3 |
| Example 2 | | |
| Comparative | 725° C. | 80.5 |
| Example 6: | | |
| Ag—Mn—Na2WO4/ | | |
| SiO2 (Catalyst #6 in | | |
| US20170014807) | | |

From Table 10, it is evident that the inventive catalyst composition of Example 2 achieves 90% oxygen conversion at a significantly lower temperature, which is almost 26% lower than the temperature at which 90% oxygen conversion occurs for the catalyst composition of Example 6, even though 5 times the catalyst loading was used for the purposes of comparative Example 6 when compared with the catalyst loading of Example 2. This finding signifies a much improved catalyst activity for the inventive composition of Example 2 over that of the composition of Example 6 along with a significantly improved catalyst loading efficiency. Further, it may be appreciated, that the selectivity of the catalyst of Example 2 is similar to that of Example 6, thereby indicating little or no deterioration in selectivity of the inventive composition of Example 2, even though the inventive catalysts demonstrated significant increase in catalyst activity. Thus, it may be concluded that although silver promoted oxidative coupling of methane catalyst was known by way of the invention described in US20170014807, the present invention provides silver promoted oxidative coupling of methane catalysts with significantly higher catalyst activity while retaining sufficient or even comparable C₂₊ hydrocarbon selectivity.

Summary—

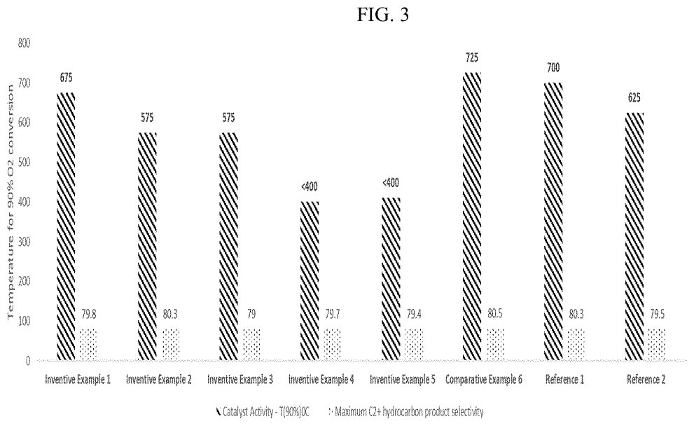

FIG. 3, provides a graphical overview of the catalyst activity expressed by way of (T(90%)° C.) value and the C₂₊ hydrocarbon selectivity, for each of the inventive composition and of the controls including comparative Example 6. Based on the results obtained by the practice of the inventive Examples 1-5, it is evident that the incorporation of silver (Ag) promoter, creates a catalyst composition having improved catalyst activity over that of the corresponding catalyst composition free of silver promoter. In particular, the use of silver nanoparticles imparts significant catalyst activity as evidenced from the (T(90%)° C.) value obtained by the practice of Example 4 and Example 5. Specifically inventive catalyst compositions prepared using silver nanoparticle precursors showed at least 28% lower (T(90%)° C.) value with minimal impact or deterioration on C₂₊ hydrocarbon selectivity. Although catalyst compositions prepared using silver salts such as silver nitrate, showed improved catalyst activity, the degree of improvement in catalyst activity using silver nanoparticles is far more significant. The improved catalyst reactivity is particularly beneficial, in terms of improving catalyst load efficiency, and eliminating the need of heat exchangers. Further as evidenced from the comparative Example 6, the silver promoted catalyst composition of the present invention demonstrates improved catalyst activity even when compared and contrasted with previously disclosed silver promoted catalyst composition such as the catalyst compositions described in the US patent publication US20170014807.

1. A composition, comprising a catalyst represented by a general formula (I):
(Ag_{z}AEₐRE1_{b}RE2_{c}AT_{d}O_{X})
wherein,
(i) ‘Ag’ represents silver;
(ii) ‘AE’ represents an alkaline earth metal;
(iii) ‘RE1’ represents a first rare earth element;
(iv) ‘RE2’ represents a second rare earth element; and
(v) ‘AT’ represents a third rare earth element ‘RE3’, or a redox agent selected from antimony, tin, nickel, chromium, molybdenum, tungsten; wherein, ‘a’, ‘b’, ‘c’, ‘d’ and ‘z’ represents relative molar ratio; wherein ‘a’ is 1; ‘b’ ranges from about 0.1 to about 10; ‘c’ ranges from about 0.01 to about 10; ‘d’ ranges from 0 to about 10; ‘z’ ranges from about 0.01 to about 1; ‘x’ balances the oxidation state; wherein, the first rare earth element, the second rare earth element and the third rare earth element, are different.

2. The composition of claim 1, wherein the relative molar ratio ‘z’ ranges from about 0.04 to about 0.18.

3. The composition of claim 1, wherein the relative molar ratio ‘a’ is 1, the relative molar ratio ‘b’ is 0.9, the relative molar ratio ‘c’ is 0.7, the relative molar ratio ‘d’ is 0.1, and the relative molar ratio ‘z’ ranges from about 0.043 to about 0.093.

4. The composition of claim 1, wherein the alkaline earth metal ‘AE’ is selected from the group consisting of magnesium, calcium, strontium, barium, and combinations thereof.

5. The composition of claim 1, wherein the first rare earth element (RE1), the second rare earth element (RE2), and the third rare element (RE3) are each independently selected from the group consisting of lanthanum, scandium, cerium, praseodymium, neodymium, promethium, samarium, europium, gadolinium, yttrium, terbium, dysprosium, holmium, erbium, thulium, ytterbium, lutetium, and combinations thereof.

6. The composition of claim 1, wherein alkaline earth metal ‘AE’ is strontium, first rare earth element ‘RE’ is lanthanum, second rare earth element ‘RE2’ is neodymium, third rare earth element ‘RE3’ is ytterbium.

7. The composition of claim 1, wherein the catalyst has a formula represented by Ag_{0.046}Sr_{1.0}La_{0.9}Nd_{0.7}Yb_{0.1}Oₓ wherein the relative molar ratio ‘z’ is 0.046, the relative molar ratio ‘a’ is 1, the relative molar ratio ‘b’ is 0.9, the relative molar ratio ‘c’ is 0.7, and the relative molar ratio ‘d’ is 0.1.

8. The composition of claim 1, wherein the catalyst has a formula represented by Ag_{0.091}Sr_{1.0}La_{0.9}Nd_{0.7}Yb_{0.1}Oₓ wherein the relative molar ratio ‘z’ is 0.091, the relative molar ratio ‘a’ is 1, the relative molar ratio ‘b’ is 0.9, the relative molar ratio ‘c’ is 0.7, and the relative molar ratio ‘d’ is 0.1.

9. The composition of claim 1, wherein the catalyst has a formula represented by Ag_{0.083}Sr_{1.0}La_{0.9}Nd_{0.7}Yb_{0.1}Oₓ wherein the relative molar ratio ‘z’ is 0.083, the relative molar ratio ‘a’ is 1, the relative molar ratio ‘b’ is 0.9, the relative molar ratio ‘c’ is 0.7, and the relative molar ratio ‘d’ is 0.1.

10. The composition of claim 1, wherein the composition has a 90% oxygen conversion temperature (T(90%)° C.) ranging from about 200° C. to about 700° C., when the composition is used in a process for producing C₂₊ hydrocarbon mixture product from methane and oxygen.

11. The composition of claim 1, wherein the composition has a C₂₊ hydrocarbon selectivity ranging from about 70% to about 88% of total product formed, when the composition is used in a process for producing C₂₊ hydrocarbon mixture product from methane and oxygen.

12. A method for preparing the composition of claim 1, the method comprising:
(i) forming an aqueous catalyst precursor solution comprising a silver agent and a precursor mixture comprising an alkaline earth metal compound and at least two rare earth metal compounds;
(ii) drying the aqueous catalyst precursor solution at a temperature of at least 90° C. and forming a dried catalyst precursor mixture; and
(iii) calcining the dried catalyst precursor mixture for at least 5 hours at a temperature of at least 650° C. and forming the composition.

13. The method of claim 12, wherein the method further comprises calcining the precursor mixture and forming a calcined precursor mixture.

14. The method of claim 12, wherein the silver agent is selected from the group consisting of silver nanoparticles, silver nanowires, silver salts and combinations thereof.

15. The method of claim 12, wherein the silver agent is a silver nanoparticle.

16. The method of claim 12, wherein the silver agent is a silver salt.

17. A process for producing a C₂₊ hydrocarbon mixture product comprising:
(a) introducing a feed mixture comprising methane and oxygen in a reactor containing the composition of claim 1;
(b) subjecting the feed mixture to a methane coupling reaction under conditions suitable to produce the C₂₊ hydrocarbon mixture product; and
(c) recovering the C₂₊ hydrocarbon mixture product after removing unconverted methane and steam from the C₂₊ hydrocarbon mixture product.

18. The process of claim 17, wherein methane to oxygen ratio ranges from about 2:1 to about 15:1.

19. The process of claim 17, wherein the feed mixture comprising methane and oxygen is introduced in the reactor at a feed temperature of less than 400° C.

20. The process of claim 17, wherein the C₂₊ hydrocarbon mixture product is produced at a reactor temperature ranging from about 300° C. to about 800° C.
